(19) United States
(12) Patent Application Publication
(10) Pub. No.: US 2008/0221361 A1
(43) Pub. Date: Sep. 11, 2008

(54) Ionic Liquids

(75) Inventor: Adam John Walker, Lincolnshire (GB)

Correspondence Address: POLSINELLI SHALTON FLANIGAN SUELTHAUS PC, 700 W. 47TH STREET, SUITE 1000, KANSAS CITY, MO, 64112-1802, US

(73) Assignee: BIONIQS LIMITED, York (GB)

(21) Appl. No.: 12/088,509

(22) PCT Filed: Sep. 28, 2006
(86) PCT No.: PCT/GB2006/003586

§ 371 Date: May 2, 2008

(30) Foreign Application Priority Data
Sep. 30, 2005 (GB) 0519898.1

(51) Int. Cl. C07C 217/08; C07C 217/28

(52) U.S. Cl. 564/508; 564/503

(57) ABSTRACT

The invention provides ionic liquids and processes for their preparation. The liquids may either comprise a cation of the formula (I); N⁺HR¹R²R³ (I) wherein R¹ is a group —R⁴—O—R⁵; R² and R³ are each independently either hydrogen or hydrocarbyl, R² and R³ may be joined together with the N to form a heterocyclic group; R⁴ is a divalent hydrocarbyl radical; and R⁵ is hydrocarbyl; or a cation of the formula (II), N+HR6R7R8 (II) wherein R⁶ is an alkanolyl group; R⁷ is a hydrocarbyl group; and R⁸ is either hydrogen or hydrocarbyl, or R⁷ and R⁸ may be joined together with the N to form a heterocyclic group.

### FIELD OF THE INVENTION

The present invention relates to ionic liquids and uses thereof. The invention also provides processes for the manufacture of ionic liquids.

### BACKGROUND TO THE INVENTION

Ionic liquids are compounds which are composed exclusively or predominantly of ions but are in liquid form, generally having a melting point below ambient temperature. They arise from combinations of suitable ions, in which the lattice energy and melting point are abnormally low. This may be achieved through the use of bulky, asymmetrical, charge-delocalised ions, which associate relatively weakly and with a low degree of structural order.

Ionic liquids can possess a number of remarkable properties, including negligible vapour pressure, high solubilising power and a broad liquid temperature range, which have rendered them interesting alternatives to conventional liquids in a variety of applications.

Ionic liquids may be made up of anions and cations or alternatively consist of zwitterions which carry both a positive and a negative charge on the same molecule. Most commonly an ionic liquid will comprise an anion and a cation.

Early ionic liquids comprised nitrogen- or phosphorous-based cations, generally substituted with one or more alkyl groups. Examples were based on a nucleus selected from quaternary ammonium cations, pyrrolidinium cations, imidazolium cations, triazolium cations, pyridinium cations, pyridazinium cations, pyrimidinium cations, pyrazinium cations and triazinium cations. These types of ionic liquids tend to be highly viscous, potentially hazardous and strongly absorbent of UV and visible light. Furthermore, the preparation of these ionic liquids can involve a number of chemical and chromatographic steps that can make the process time consuming, expensive and inefficient.

In WO-2004/063383, modified ionic liquids were disclosed in which one of the component ions, typically the cation, included a functional group selected from alkenyl, hydroxyl, amino, thio, carbonyl and carboxyl groups. By modifying the liquids in this way, it was found possible to tailor them for use as solvents in various applications, in particular for single-phase biocatalysis. The liquids could be made more biocompatible, and could provide a more polar, protic, hydrogen bonding environment to mimic that which would previously have been achieved using aqueous solvents. Thus, enzyme-catalysed reactions that could not previously be carried out in non-aqueous environments could now be performed in ionic liquids, with all their associated advantages.

Our co-pending PCT patent application no. PCT/GB2005/001364 discloses further ionic liquids which comprise as the cation a primary, secondary or tertiary ammonium ion containing a protonated nitrogen atom. The nitrogen atom can be substituted with one, two or three hydrocarbyl groups, and the hydrocarbyl groups can themselves be substituted, in order to tailor their functionality, with groups such as nitrogen-containing functional groups (including nitrile, nitro or amino or another basic nitrogen-containing functional group), thiol, alkylthio, sulphonyl, thiocyanate, isothiocyanate, azido, hydrazino, halogen, alkyl optionally interrupted by one or more ether or thioether linkages, alkoxy, alkenyl, hydroxy, carbonyl, carboxyl, boronate, silyl and substituted amino. Such liquids have been found to demonstrate high solvation capabilities, low viscosity and low toxicity, making them useful in a broader range of applications than some of the previously available ionic liquids.

Anderson et al, J. Am. Chem. Soc. 124:14247-14254 (2002) also disclose ionic liquids composed of a primary or tertiary ammonium based cation for use in certain chemical applications.

Ionic liquids containing a hydroxyl group —OH on one of the hydrocarbyl side chains have been used, as described in WO-2004/063383, as reaction media for biocatalytic reactions. In some situations, however, for example when the enzyme used is a hydrolase, such ionic liquids can suffer from the drawback that the hydroxyalkyl function may interfere with or participate in the reaction being catalysed.

The present inventors have developed alternative ionic liquids, which can be used as solvents and as reaction media in a wide range of situations, including those in which a hydrogen bonding, protic environment is required, and including for biological solutes such as enzymes. The invention can thus broaden the range of applications for ionic liquids, in particular as solvents and/or reaction media and more particularly in biocatalysis.

### STATEMENTS OF THE INVENTION

According to a first aspect of the present invention there is provided an ionic liquid comprising a cation of the formula (I):

N⁺H¹R²R³ (I)

wherein R¹ is a group —R⁴—O—R⁵;

R² and R³ are each independently either hydrogen or hydrocarbyl, or R² and R³ may be joined together with the N to form a heterocyclic group;

R⁴ is a divalent hydrocarbyl radical; and

R⁵ is hydrocarbyl.

Ionic liquids according to this first aspect of the invention, which contain both a labile proton (on the central nitrogen atom) and an ether group —R⁴—O—R⁵, have been found to be capable of hydrogen bonding and hence of providing a fluid environment which is similar in functional terms to that of an aqueous solvent. They can thus be used as solvents and reaction media for relatively hydrophilic materials, in particular for enzymes and enzyme-catalysed reactions.

A further advantage of such ionic liquids is their ability to provide a polar, hydrogen bonding environment in the absence of hydroxyl groups. This can help to overcome the drawbacks referred to above, where the presence of a hydroxyl moiety on an ionic liquid solvent can in cases react with a solute such as an activated acid or a strong base, or interfere with a reaction (in particular an enzyme-catalysed reaction such as one involving a hydrolase or esterase) being carried out in the ionic liquid.

The cation (I) may be a primary ammonium ion, in which R² and R³ are both hydrogen. It may be a secondary ammonium ion, in which only one of R² and R³ is hydrogen. It may be a tertiary ammonium ion, in which neither of R² and R³ is hydrogen. Preferably, it is a secondary or a tertiary ammonium ion. Tertiary ions may be particularly preferred, since they tend to be less reactive than their primary or secondary counterparts, and can be less likely to form unwanted and potentially toxic byproducts such as nitrosamines. The presence of at least one labile proton on the nitrogen atom is however desirable as it tends to lower the viscosity of the ionic liquid and also helps to provide the protic, hydrogen bonding environment which makes the ionic liquid suitable for use as a solvent for hydrophilic materials.

In the context of the present invention, a hydrocarbyl group may be substituted with one or more substituents selected from nitrogen-containing functional groups (including nitrile, nitro or amino or another basic nitrogen-containing functional group), thiol, alkythio, sulphonyl, thiocyanate, isothiocyanate, azido, hydrazino, halogen, alkyl, alkyl interrupted by one or more ether or thioether linkages, alkoxy, alkenyl, hydroxy, carbonyl (including aldehyde or ketone), carboxyl, boronate, silyl and substituted amino (eg, mono- or di-alkylamino or alkylamido).

Preferred substituents for use in this context are selected from the group consisting of alkenyl, hydroxyl, alkoxy, amino, thio, carbonyl and carboxyl groups. More preferably, substituents are selected from hydroxyl and amino groups; yet more preferably a substituent is a hydroxyl group.

Preferably, however, in the context of the present invention, a hydrocarbyl group is unsubstituted.

Preferably R⁴ is —(CH₂)ₙ—, where n is an integer from 2 to 8, preferably from 2 to 6, more preferably from 2 to 4, such as 2 or 3, suitably 2.

It may be preferred, in particular if R² and R³ are both hydrogen and R⁵ is an unsubstituted alkyl group, for R⁴ not to be CH₂CH₂. In other words, it may be preferred for the cation (I) not to be an alkoxyethyl ammonium cation.

R¹ may for example be a methoxyethyl group, in particular when R² and R³ are not both hydrogen.

Preferably R⁵ is alkyl or cycloalkyl, more preferably C₁ to C₆ alkyl or cycloalkyl, yet more preferably C₁ to C₅ alkyl, such as in particular methyl, ethyl, propyl, iso-propyl, butyl, iso-butyl or tert-butyl, more particularly methyl or ethyl, suitably methyl. Preferably R⁵ is either unsubstituted or is substituted with a hydroxyl group, in particular a terminal hydroxyl group.

Thus in some cases it may be preferred for R⁵ to be an unsubstituted alkyl group such as CH₃ or (CH₂)ₙCH₃, with n being an integer for example from 1 to 4, preferably either 1 or 2 and most preferably 1. In other cases it may be preferred for R⁵ to be (CH₂)ₙOH, with n being an integer suitably from 2 to 4, preferably either 2 or 3 and most preferably 2. This latter case, where R¹ is a (hydroxyalkoxy)alkyl group, may be particularly preferred when R² and R³ are both alkyl groups, in particular selected from methyl and ethyl groups, most particularly methyl groups; thus, the cation (I) may be a N,N-dialkyl-N-[(hydroxyalkoxy)alkyl] ammonium ion such as a N,N-dimethyl-N-[(hydroxyalkoxy)alkyl] ammonium ion, in particular a N,N-dimethyl-N-[(2-hydroxyethoxy)ethyl] ammonium ion.

It may be preferred for R¹ not to be a methoxyethyl group, in particular if both R² and R³ are hydrogen.

It may be preferred for R¹ not to be a methoxypropyl group, in particular if both R² and R³ are hydrogen.

In some cases it may be preferred for R¹ not to be an alkoxyethyl group, in particular if both R² and R³ are hydrogen.

Preferably R² is alkyl or cycloalkyl, more preferably C₁ to C₆ alkyl or cycloalkyl, yet more preferably C₁ to C₅ alkyl, such as in particular methyl, ethyl, propyl, iso-propyl, butyl, iso-butyl or tert-butyl, more particularly methyl, ethyl, propyl or iso-propyl, yet more particularly methyl or ethyl. Preferably R² is unsubstituted.

R² may be a group of formula —R⁴—O—R⁵, where R⁴ and R⁵ are as defined above; in this case, R² may be the same as or different to R¹. In particular, R¹ may be the same as R², and may for example be selected from methoxyalkyl and alkoxyethyl, in particular (so long as R³ is not hydrogen) methoxyethyl.

In some cases it may be preferred for R² to be hydrogen.

Preferably R³ is hydrogen. In some cases however it may be an alkyl or cycloalkyl group, for instance as defined above in connection with R².

In an embodiment of the invention, for example, R³ is an alkyl group and R¹ and R² are both alkoxyalkyl groups of the formula —R⁴—O—R⁵. In this case R³ may be for instance a C₁ to C₆ alkyl group, preferably a C₁ to C₅ alkyl group, such as in particular methyl, ethyl, propyl, iso-propyl, butyl, iso-butyl or tert-butyl, more particularly methyl or ethyl, suitably methyl; and R¹ and R² are preferably the same and may be of the types defined above, in particular selected from methoxyalkyl and alkoxyethyl, more particularly methoxyethyl.

In another embodiment, R² and R³ may both be alkyl or cycloalkyl, for instance as defined above in connection with R². In this case R² and R³ are preferably both alkyl, more preferably C₁ to C₃ alkyl; they may be the same or different, preferably the same. The presence of two alkyl groups, in particular lower alkyl groups such as ethyl or in particular methyl, has been found to help lower the viscosity of the ionic liquid which can be advantageous in the context of its use as a solvent or reaction medium.

Thus, the cation (I) may for instance be a dialkyl alkoxyalkyl ammonium ion, preferably a dimethyl, diethyl or dipropyl alkoxyalkyl ammonium ion, a methyl ethyl alkoxyalkyl ammonium ion, a methyl propyl alkoxyalkyl ammonium ion or an ethyl propyl alkoxyalkyl ammonium ion. Most preferably (I) is selected from dimethyl alkoxyalkyl, diethyl alkoxyalkyl and methyl ethyl alkoxyalkyl ammonium ions, in particular dimethyl alkoxyalkyl ammonium. In such a case R¹ could be for example a methoxyethyl group.

Preferably R³ is not the same as R¹.

In an embodiment of the invention, R³ may be an alkanolyl group, for example as defined in connection with formula (II) below, in particular if R² is an alkyl group such as a C₁ to C₆ or C₁ to C₄ alkyl group.

R² is preferably not the same as R¹, in particular if R³ is hydrogen. It may be preferred, again particularly if R³ is hydrogen, for R¹ and R² not both to be alkoxyalkyl groups, or at least for R¹ and R² not to be the same alkoxyalkyl group. In some cases it may be preferred, particularly if R³ is hydrogen, for R² not to be an alkoxyalkyl group.

In particular if R³ is hydrogen, and more particularly if R³ is hydrogen and R¹ is methoxyethyl, R² is preferably not methoxyethyl.

Particularly preferred ionic liquids according to the invention comprise a cation of the formula (Ia):

N⁺HR¹R²R³ (Ia)

wherein R¹ is a group —R⁴—O—R⁵;

R² and R³ are each independently either hydrogen, alkanolyl, alkyl or a group —R⁴—O—R⁵, preferably either hydrogen, alkyl or —R⁴—O—R⁵, more preferably either hydrogen or alkyl;

R⁴ is unsubstituted alkylene, more preferably —(CH₂)_{N} where n is as defined above; and

R⁵ is alkyl or a group (CH₂)ₙOH (where n is an integer suitably from 2 to 4), preferably alkyl.

Other preferred features of the substituents of the cation (Ia) may be as defined above for the cation (I).

Preferably the cation (Ia) is not an alkoxyethyl ammonium ion or a di(alkoxyalkyl) ammonium ion (in particular not a di(methoxyethyl) ammonium ion).

Preferably the cation (Ia) is a secondary or tertiary ammonium ion, more preferably tertiary. Suitably each alkyl, alkylene and alkanoyl group is independently selected from groups containing from 1 to 4, preferably from 1 to 3, carbon atoms.

Particularly preferred ionic liquids according to the first aspect of the invention comprise a cation selected from alkoxypropyl (preferably methoxypropyl) ammonium ions, methoxyalkyl ammonium ions (preferably other than methoxyethyl ammonium ions), di(alkoxyalkyl) ammonium ions other than di(methoxyethyl) ammonium ions, alkyl alkoxyalkyl ammonium ions (preferably methyl alkoxyalkyl or alkyl methoxyethyl ammonium ions), dialkyl alkoxyalkyl ammonium ions (preferably dimethyl alkoxyalkyl or dialkyl methoxyethyl ammonium ions), alkyl di(alkoxyalkyl) ammonium ions (preferably methyl di(alkoxyalkyl) ammonium ions or alkyl di(methoxyethyl) ammonium ions) and N,N-dialkyl-N-[(hydroxyalkoxy)alkyl] ammonium ions.

Yet more preferred ionic liquids according to the first aspect of the invention comprise a cation selected from alkyl alkoxyalkyl ammonium ions, dialkyl alkoxyalkyl ammonium ions, alkyl di(alkoxyalkyl) ammonium ions and N,N-dialkyl-N-[(hydroxyalkoxy)alkyl] ammonium ions.

Most preferred ionic liquids according to the first aspect of the invention comprise a cation selected from dialkyl alkoxyalkyl ammonium ions, alkyl di(alkoxyalkyl) ammonium ions and N,N-dialkyl-N-[(hydroxyalkoxy)alkyl] ammonium ions.

It may be preferred for the ionic liquid of the first aspect of the present invention not to be any of the following compounds:
N,N-Di(methoxyethyl)ammonium chloride
N,N-Di(methoxyethyl)ammonium bromide
N,N-Di(methoxyethyl)ammonium iodide
N,N-Di(methoxyethyl)ammonium formate
N,N-Di(methoxyethyl)ammonium acetate
N,N-Di(methoxyethyl)ammonium propanoate
N,N-Di(methoxyethyl)ammonium propanedioate
N,N-Di(methoxyethyl)ammonium butanoate
N,N-Di(methoxyethyl)ammonium butenoate
N,N-Di(methoxyethyl)ammonium butanedioate
N,N-Di(methoxyethyl)ammonium pentanoate
N,N-Di(methoxyethyl)ammonium pentanedioate
N,N-Di(methoxyethyl)ammonium pentenoate
N,N-Di(methoxyethyl)ammonium hexanoate
N,N-Di(methoxyethyl)ammonium hexenoate
N,N-Di(methoxyethyl)ammonium heptanoate
N,N-Di(methoxyethyl)ammonium heptanedioate
N,N-Di(methoxyethyl)ammonium heptenoate
N,N-Di(methoxyethyl)ammonium octanoate
N,N-Di(methoxyethyl)ammonium octanedioate
N,N-Di(methoxyethyl)ammonium octenoate
N,N-Di(methoxyethyl)ammonium nonanoate
N,N-Di(methoxyethyl)ammonium nonanedioate
N,N-Di(methoxyethyl)ammonium nonenoate
N,N-Di(methoxyethyl)ammonium decanoate
N,N-Di(methoxyethyl)ammonium decanedioate
N,N-Di(methoxyethyl)ammonium decenoate
N,N-Di(methoxyethyl)ammonium undecanoate
N,N-Di(methoxyethyl)ammonium undecanedioate
N,N-Di(methoxyethyl)ammonium undecenoate
N,N-Di(methoxyethyl)ammonium dodecanoate
N,N-Di(methoxyethyl)ammonium dodecanedicarboxylate
N,N-Di(methoxyethyl)ammonium dodecenecarboxylate
N,N-Di(methoxyethyl)ammonium cyclohexanecarboxylate
N,N-Di(methoxyethyl)ammonium cyclohexenecarboxylate
N,N-Di(methoxyethyl)ammonium phenoxide
N,N-Di(methoxyethyl)ammonium benzoate
N,N-Di(methoxyethyl)ammonium benezenedicarboxylate
N,N-Di(methoxyethyl)ammonium benzenetricarboxylate
N,N-Di(methoxyethyl)ammonium benzenetetracarboxylate
N,N-Di(methoxyethyl)ammonium chlorobenzoate
N,N-Di(methoxyethyl)ammonium fluorobenzoate
N,N-Di(methoxyethyl)ammonium pentachlorobenzoate
N,N-Di(methoxyethyl)ammonium pentafluorobenzoate
N,N-Di(methoxyethyl)ammonium salicylate
N,N-Di(methoxyethyl)ammonium glycolate
N,N-Di(methoxyethyl)ammonium lactate
N,N-Di(methoxyethyl)ammonium pantothenate
N,N-Di(methoxyethyl)ammonium tartrate
N,N-Di(methoxyethyl)ammonium hydrogen tartrate
N,N-Di(methoxyethyl)ammonium mandelate
N,N-Di(methoxyethyl)ammonium crotonate
N,N-Di(methoxyethyl)ammonium malate
N,N-Di(methoxyethyl)ammonium pyruvate
N,N-Di(methoxyethyl)ammonium succinate
N,N-Di(methoxyethyl)ammonium citrate
N,N-Di(methoxyethyl)ammonium fumarate
N,N-Di(methoxyethyl)ammonium phenylacetate
N,N-Di(methoxyethyl)ammonium oxalate
N,N-Di(methoxyethyl)ammonium bis(trifluoromethylsulphonyl)imide
N,N-Di(methoxyethyl)ammonium carbonate
N,N-Di(methoxyethyl)ammonium hydrogen carbonate
N,N-Di(methoxyethyl)ammonium sulphate
N,N-Di(methoxyethyl)ammonium hydrogen sulphate
N,N-Di(methoxyethyl)ammonium phosphate
N,N-Di(methoxyethyl)ammonium hydrogen phosphate
N,N-Di(methoxyethyl)ammonium dihydrogen phosphate
N,N-Di(methoxyethyl)ammonium methanesulphonate
N,N-Di(methoxyethyl)ammonium trifluoromethanesulphonate
N,N-Di(methoxyethyl)ammonium ethylenediaminetetraacetate
N,N-Di(methoxyethyl)ammonium hexafluorophosphate
N,N-Di(methoxyethyl)ammonium tetrafluoroborate
N,N-Di(methoxyethyl)ammonium trifluoroacetate
N,N-Di(methoxyethyl)ammonium pentafluoropropanoate
N,N-Di(methoxyethyl)ammonium heptafluorobutanoate
N,N-Di(methoxyethyl)ammonium phosphoenolpyruvate
N,N-Di(methoxyethyl)ammonium nicotinamide adenine dinucleotide phosphate
N,N-Di(methoxyethyl)ammonium adenosinephosphate
N,N-Di(methoxyethyl)ammonium adenosine diphosphate
N,N-Di(methoxyethyl)ammonium adenosine triphosphate
N,N-Di(methoxyethyl)ammonium oxyniacate
N,N-Di(methoxyethyl)ammonium nitrate
N,N-Di(methoxyethyl)ammonium nitrite.

However, generally speaking any of the anions referred to in this list may be used as the counterion in an ionic liquid according to the invention.

In another embodiment of the invention, either or both of R² and R³ may be substituted with one or more hydroxyl groups, preferably one; it may for example be an alkanolyl such as a C₂ to C₆, preferably a C₂ to C₅, alkanolyl, in particular ethanolyl, propanolyl or butanolyl, more particularly ethanolyl or propanolyl. Such groups may be substituted with two or more, such as two or three, hydroxyl groups; they may thus contain diol or polyol moieties. Preferably such a group has a terminal hydroxyl group, such as in an ethanolyl or 3-hydroxypropyl group. In this embodiment of the invention, R² and R³ may again be the same or different, preferably the same.

Suitably R² and R³ are not joined together with the N to form a heterocyclic group. If they are, the heterocyclic group is preferably not a heteroaryl group; in particular the cation (I) is preferably not a pyridinium, pyrrolidinium or imidazolium cation.

Preferably R² and R³ are not both alkoxyalkyl. More preferably neither R² nor R³ is alkoxyalkyl. An alkoxyalkyl group typically means a group of the formula —R⁴—O—R⁵ where R⁴ and R⁵ are both unsubstituted alkyl groups.

The term “ionic liquid” herein includes, but is not limited to, a compound consisting of ions and liquid at temperatures at which the compound is stable. An “ionic liquid” must be a compound composed of ions, including a stable stoichiometric hydrate or other solvate of such an ionic material. It need not necessarily be composed exclusively of ions; it may for example exist as an equilibrium mixture of ions and molecules although at least some of the liquid must be present in ionic form.

Ionic liquids typically have a freezing point below 100° C. Suitably an ionic liquid according to the invention will be capable of existing in liquid form at and below 50° C., preferably at and below 40° C., more preferably at and below 30° C. and ideally at room temperature, which for the present purposes may be defined as from 18 to 25° C., typically about 20° C. Its boiling point may be at least 200° C., in cases above 500° C.

An ionic liquid according to the invention may thus consist substantially of ions, and is preferably liquid at the above defined temperatures in the dry state. Such ionic liquids will generally contain 5% or less of water, by mass, preferably 1% or less or 1000 ppm or less and more preferably 100 ppm or less.

Preferably an ionic liquid according to the invention has a viscosity of less than 500 centipoise at 25° C.

In the present context, “hydrocarbyl” may be defined as any group containing carbon and hydrogen, which may also contain one or more heteroatoms such as oxygen, nitrogen, sulphur, phosphorous or halogen. The term embraces saturated, partially saturated and unsaturated groups, whether aromatic or aliphatic, whether straight chain, branched chain, cyclic or any combination thereof. Hydrocarbyl thus includes, but is not limited to, optionally substituted alkyl, alkenyl, alkynyl, cycloalkyl, cycloalkenyl, cycloalkynyl, aryl, aralkyl, alkaryl, heterocyclyl, heteroaryl, alkoxy and moieties containing a combination of two or more such groups.

In the present context, a hydrocarbyl group preferably does not contain heteroatoms. It is preferably aliphatic.

As used herein, “alkyl” includes both straight and branched chain alkyl radicals, of any chain length but typically of from 1 to 12 carbon atoms, more suitably from 1 to 10 or from 1 to 8 carbon atoms, preferably from 1 to 6 carbon atoms. Suitable examples of alkyl groups include methyl, ethyl, n-propyl, isopropyl, n-butyl, sec-butyl, isobutyl and tert-butyl. The term “cycloalkyl” encompasses aliphatic saturated hydrocarbyl ring-containing moieties such as for example cyclopropyl, cyclobutyl, cyclopentyl or cyclohexyl.

The term “alkenyl” includes both straight and branched chain alkenyl radicals, which contain one or more carbon-carbon double bonds. Again they may be of any chain length, typically from 2 to 12 carbon atoms, more suitably from 2 to 10 or from 2 to 8 carbon atoms, yet more preferably from 2 to 6 carbon atoms. Examples include ethylene, n-propyl-1-ene, n-propyl-2-ene and isopropylene.

“Cycloalkenyl” encompasses ring-containing groups where the ring structure incorporates one or more carbon-carbon double bonds.

The term “alkynyl” includes both straight and branched chain alkynyl radicals, which contain one or more carbon-carbon triple bonds. They may be of any chain length, typically from 2 to 12 carbon atoms, more suitably from 2 to 10 or from 2 to 8 carbon atoms, yet more preferably from 2 to 6 carbon atoms. “Cycloalkynyl” encompasses ring-containing groups where the ring structure incorporates one or more carbon-carbon triple bonds.

The term “aryl” includes aromatic (and thus at least partially unsaturated) hydrocarbyl groups, which will typically incorporate one or more cyclic structures. Such groups may contain for example from 3 to 12 carbon atoms, preferably from 3 to 10 or from 4 to 8 carbon atoms. They may be fused to one or more saturated or unsaturated rings. A typical example is phenyl. It is to be noted that the term “hydrocarbyl” also embraces radicals which combine both alkyl and aryl moieties, in particular aralkyl and alkaryl groups such as for instance benzyl.

The term “heterocyclyl” includes a ring system containing one or more heteroatoms selected for example from N, O and S. It may be saturated, unsaturated or partially unsaturated. The ring containing the heteroatom may be fused to one or more other rings, which in turn may be saturated, unsaturated or partially unsaturated and may themselves contain heteroatom(s). Typically a heterocyclyl radical will be a 3 to 10-membered ring system, preferably a 5 to 10-membered system, more preferably a 5- or 6-membered system. It may be or incorporate aromatic moieties.

Examples of cyclic groups such as cycloalkyl, aryl or heterocyclyl include but are not limited to cyclohexyl, phenyl, acridine, benzimidazole, benzofuran, benzothiophene, benzoxazole, benzothiazole, carbazole, cinnoline, dioxin, dioxane, dioxolane, dithiane, dithiazine, dithiazole, dithiolane, furan, imidazole, imidazoline, imidazolidine, indole, indoline, indolizine, indazole, isoindole, isoquinoline, isooxazole, isothiazole, morpholine, napthyridine, oxazole, oxadiazole, oxathiazole, oxathiazolidine, oxazine, oxadiazine, phenazine, phenothiazine, phenoxazine, phthalazine, piperazine, piperidine, pteridine, purine, putrescine, pyran, pyrazine, pyrazole, pyrazoline, pyrazolidine, pyridazine, pyridine, pyrimidine, pyrrolidine, pyrrole, pyrroline, quinoline, quinone, quinoxaline, quinazoline, quinolizine, tetrahydrofuran, tetrazine, tetrazole, thiophene, thiadiazine, thiadiazole, thiatriazole, thiazine, thiazole, thiomorpholine, thianaphthalene, thiopyran, triazine, triazole, trithiane and tropine.

The term “alkoxy” includes both straight chain and branched alkyl radicals, for example of 1 to 12 carbon atoms, preferably of 1 to 8 or 1 to 6 or 1 to 4 or 1 to 3 carbon atoms, which contain one or more oxygen atoms, typically in the form of a hydrocarbyl group linked to an oxygen atom via an ether linkage. Examples include methoxy and ethoxy groups.

The term “halogen” means either F, Cl, Br or I, typically either F, Cl or Br, more typically either F or Cl.

An ionic liquid according to the present invention preferably comprises an anion, for example a counterion X^{m-} where m is an integer such as in particular 1, 2 or 3, preferably 1 or 2, most typically 1. This may be any suitable anion; the only theoretical constraint upon the choice of anion is its ionic weight in order to keep the freezing point of the ionic liquid below the desired temperature.

Examples of suitable anions include halogenated inorganic or organic anions, nitrates, sulphates, phosphates, carbonates, sulphonates and carboxylates. The sulphonates and carboxylates may be alkylsulphonates and alkylcarboxylates, in which the alkyl group is a moiety, for example having 1 to 20 carbon atoms, selected from alkyl and alkyl substituted at any position with alkenyl, alkoxy, alkeneoxy, aryl, arylalkyl, aryloxy, amino, aminoalkyl, thio, thioalkyl, hydroxyl, hydroxyalkyl, carbonyl, oxoalkyl, carboxyl, carboxyalkyl or halogen, including all salts, ethers, esters, pentavalent nitrogen or phosphorous derivatives or stereoisomers thereof.

For example, the anion may be selected from bis(trifluoromethylsulphonyl)imide, carbonate, hydrogen carbonate, sulphate, hydrogen sulphate, sulphite, hydrogen sulphite, silicate, phosphate, hydrogen phosphate, dihydrogen phosphate, hydrogen phosphite, dihydrogen phosphite, metaphosphate, methanesulphonate, ethanesulphonate, benzenesulphonate, trifluoromethanesulphonate, ethylenediaminetetraacetate, fluoride, chloride, bromide, iodide, hexafluorophosphate, tetrafluoroborate, trifluoroacetate, pentafluoropropanoate, heptafluorobutanoate, oxalate, fornate, acetate, propanoate, butanoate, pentanoate, hexanoate, heptanoate, octanoate, nonanoate, decanoate, benzoate, benezenedicarboxylate, benzenetricarboxylate, benzenetetracarboxylate, chlorobenzoate, fluorobenzoate, pentachlorobenzoate, pentafluorobenzoate salicylate, glycolate lactate, pantothenate, tartrate, hydrogen tartrate, mandelate, acrylate, methacrylate, crotonate, malate, pyruvate, oxaloacetate, succinate, citrate, fumarate, phenylacetate, gluconate, glyoxylate, mercaptoacetate, oxamate, sulphamate, methylphosphonate, ethylphosphonate, phenylphosphonate, phenylphosphinate, thiocyanate, isothiocyanate, cyanate, isocyanate, thiosulphate, nitrate, nitrite, thiophosphate or dicyanamide.

An ionic liquid according to the invention may contain cations which are all the same or which are different. It may contain anions which are all the same or which are different. Thus the invention encompasses ionic liquids including a mixture of different cations and/or different anions.

In an ionic liquid according to the invention, the cation and anion should together be chosen to ensure that the material is liquid at the requisite temperature. Freezing point can be affected by factors such as the size of either or both of the ions, their degree of delocalisation of charge and their degree of symmetry, as described above and in the prior art literature relating to ionic liquids. The use of larger, and/or more charge-delocalised ions can for instance help to reduce the ionic liquid's freezing point.

The invention encompasses an ionic liquid which is composed not of anions and cations but of zwitterions which carry both a positive and a negative charge: in this situation, a single ion will incorporate both the moieties N⁺HR¹R²R³ and, for instance by appropriate side-chain substitution, an anionic moiety such as X^{m-}.

In addition to demonstrating high solvation capability, the ionic liquids of the present invention can have low viscosity, can be of relatively low toxicity and can be colourless. These features can make the ionic liquids of the invention useful in a variety of applications. In addition, ionic liquids of this composition can exhibit particular advantages over the corresponding hydroxyalkyl species, being effective hydrogen bond acceptors but poor donors and functioning as significantly less polar, less protic solvents.

In an ionic liquid according to the invention, the cation (I) is preferably an alkoxypropyl ammonium cation, a methyl alkoxyethyl ammonium cation, a methyl alkoxypropyl ammonium cation, a dimethyl alkoxyethyl ammonium cation, a dimethyl alkoxypropyl ammonium cation, an ethyl alkoxyethyl ammonium cation, an ethyl alkoxypropyl ammonium cation, a diethyl alkoxyethyl ammonium cation, a diethyl alkoxypropyl ammonium cation, a methyl ethyl alkoxyethyl ammonium cation, a methyl ethyl alkoxypropyl ammonium cation, a propyl alkoxyethyl ammonium cation, a propyl alkoxypropyl ammonium cation, a dipropyl alkoxyethyl ammonium cation, a dipropyl alkoxypropyl ammonium cation, a methyl propyl alkoxyethyl ammonium cation, a methyl propyl alkoxypropyl ammonium cation, an ethyl propyl alkoxyethyl ammonium cation or an ethyl propyl alkoxypropyl ammonium cation.

Of these, alkoxyethyl ammonium, alkoxypropyl ammonium, methyl alkoxyethyl ammonium, methyl alkoxypropyl ammonium, dimethyl alkoxyethyl ammonium and ethyl methyl alkoxyethyl ammonium ions may be preferred.

In all of these cations, the alkoxy group is preferably either methoxy or ethoxy.

According to a second aspect of the present invention there is provided an ionic liquid comprising a cation of the formula (II):

N⁺HR⁶R⁷R⁸ (II)

wherein R⁶ is an alkanolyl group;

R⁷ is a hydrocarbyl group; and

R⁸ is either hydrogen or hydrocarbyl,

or R⁷ and R⁸ may be joined together with the N to form a heterocyclic group.

Again, such ionic liquids have been found to be useable as solvents for hydrophilic materials, in particular for enzymes and their reactions, as they can provide a protic, hydrogen bonding fluid environment.

R⁶ may contain more than one —OH group; in other words, it may comprise a diol or polyol. It may be straight or branched chain. It preferably contains from 1 to 12 carbon atoms, more preferably from 1 to 10, yet more preferably from 1 to 8, most preferably from 1 to 6 or from 1 to 4 or from 1 to 3. Suitably R⁶ may be methanolyl, ethanolyl or propanolyl, preferably ethanolyl or propanolyl (in particular 3-hydroxylpropyl). Most suitably an alkanoyl group may be propanolyl such as 3-hydroxypropyl, 2-hydroxypropyl or propan-2,3-diolyl, in particular 3-hydroxypropyl. Preferably it contains a terminal —OH group.

R⁶ may be substituted with other groups such as those listed above as preferred hydrocarbyl substituents. Preferably R⁶ is unsubstituted other than by one or more —OH groups. In some cases, however, it may be preferred for R⁶ to contain an ether linkage—for example, R⁶ may be a (hydroxyalkoxy)alkyl group of formula —(CH₂)ₙ—O—(CH₂)ₘ—OH where n and m are independently selected integers suitably from 1 to 4, more suitably from 2 to 4, most suitably either 2 or 3, such as 2.

R⁷ is preferably an alkyl or cycloalkyl group, suitably as defined above for R². It is preferably a C₁ to C₄ alkyl group, in particular a C₁ to C₃ alkyl group, such as methyl or ethyl.

R⁷ may be an alkanolyl group, in particular as defined above for R⁶. In particular R⁶ and R⁷ may both be alkanolyl; R⁶ and R⁷ may then be different alkanolyl groups or, more preferably, the same. In one embodiment of the invention, R⁶ and R⁷ are both alkanolyl (preferably the same) and R⁸ is alkyl or cycloalkyl, suitably as defined above for R2. In another embodiment, R⁶ and R⁷ are both alkanolyl (preferably the same) and R⁸ is hydrogen.

Generally, R⁸ is preferably hydrogen. Thus, the cation (II) may for instance be an alkanolammonium ion, a dialkanolammonium ion or an alkyl alkanolammonium ion. Of these, the alkyl alkanolammonium ions (excepting in some cases the methyl ethanolammonium ions) may be preferred, in which case R⁷ may be for example a C₁ to C₄ or C₁ to C₃ alkyl group and R⁶ may be for example a C₂ to C₄ or C₂ to C₃ alkanolyl group such as ethanolyl.

However, in cases it may be preferred for R⁸ to be alkyl or cycloalkyl, suitably as defined above for R². In particular, R⁷ and R⁸ may both be alkyl or cycloalkyl, suitably as defined above in connection with R². In this case R⁷ and R⁸ are preferably both alkyl, more preferably C₁ to C₃ alkyl, yet more preferably methyl or ethyl; they may be the same or different, preferably the same. As described in connection with the first aspect of the invention, the presence of two alkyl groups can help to lower the viscosity of the ionic liquid.

Thus, the cation (II) may for instance be a dialkyl alkanolammonium ion (excepting in some cases the dimethyl ethanolammonium ions), preferably a dimethyl, diethyl or dipropyl alkanolammonium ion, a methyl ethyl alkanolammonium ion, a methyl propyl alkanolammonium ion or an ethyl propyl alkanolammonium ion. It may be a dialkyl ethanolammonium ion or a dialkyl propanolammonium ion, of which the dialkyl ethanolammonium ions may be preferred. In the case of the dialkyl ethanolammonium ions, preferably at least one of R⁷ and R⁸, and preferably both, are selected from methyl and ethyl; more preferably both are ethyl. In the case of the dialkyl propanolammonium ions, preferably at least one of R⁷ and R⁸, and preferably both, are selected from methyl and ethyl; more preferably both are methyl.

Alternatively R⁸ may be an alkanolyl group, suitably as defined above for R⁶. Thus R⁶, R⁷ and R⁸ may each independently be alkanolyl; they may be different or preferably at least two of the groups, more preferably all three, are the same.

Either or both of R⁷ and R⁸ may be independently selected from groups of the formula —R⁴—O—R⁵, for instance as defined above in connection with the first aspect of the invention. Such groups have the advantage, as described above, of providing hydrogen bonding capability but without the more reactive hydroxyl group. Suitably, R⁷ is a group of formula —R⁴—O—R⁵ and R⁸ is an alkyl group, suitably as defined above for R². Thus, the cation (II) may be an alkyl(alkoxyalkyl) alkanolyl group, in which R⁶ is preferably C₂ to C₄ alkanolyl such as ethanolyl or propanolyl, in particularly ethanolyl; R⁷ is preferably methoxy ethyl or ethoxy ethyl, more preferably the former; and R⁸ is preferably C₁ to C₄ alkyl or C₁ to C₃ alkyl, for instance methyl or ethyl, suitably methyl.

Where R⁷ is methyl, and particularly when R⁸ is hydrogen, R⁶ is preferably not ethanolyl. In other words, the cation (II) is preferably not a methyl ethanolammonium cation.

It may be preferred for the cation (II) not to be an ethyl ethanolammonium ion.

Where R⁷ is an alkanolyl group, and particularly when R⁸ is hydrogen, preferably R⁶ and R⁷ are not both ethanolyl. In other words, the cation (II) is preferably not a diethanolammonium cation. This may also be the case when R⁸ is alkyl, for instance butyl.

Where R⁷ and R⁸ are both methyl, R⁶ is preferably not ethanolyl. In other words, the cation (II) is preferably not a dimethyl ethanolammonium cation.

In cases it may be preferred for the cation (II) not to be a diethyl ethanolammonium ion.

It may be preferred for the cation not to be a dialkyl ethanolammonium cation.

Where R⁶ and R⁷ are both ethanolyl, R⁸ is preferably not alkyl. In other words, the cation (II) is preferably not an alkyl diethanolammonium cation. In particular it is preferably not a butyl diethanolammonium cation.

It may be preferred for neither of R⁷ and R⁸ to be putrescinium, in particular where the other is hydrogen. More particularly, where R⁷ is putrescinium, R⁶ is preferably not 3-hydroxypropyl, especially if R⁸ is hydrogen. In other words, the cation (II) is preferably not a 3-hydroxypropyl putrescinium cation.

Preferably R⁶, R⁷ and R⁸ are not all ethanolyl. In other words, the cation (II) is preferably not a triethanolammonium cation.

Preferably the cation (II) is not an N-(3-hydroxypropyl)-N-methylcyclohexylammonium cation.

Suitably R⁷ and R⁸ are not joined together with the N to form a heterocyclic group. If they are, the heterocyclic group is preferably not a heteroaryl group; in particular the cation (II) is preferably not a pyridinium, pyrrolidinium or imidazolium cation.

Other preferred features of this second aspect of the invention may be as defined above in connection with the first aspect. In particular, the cation (II) is preferably a secondary ammonium ion.

Particularly preferred ionic liquids according to the second aspect of the invention comprise a cation selected from alkyl alkanolammonium ions (preferably excluding methyl ethanolammonium ions) and dialkyl alkanolammonium ions (preferably excluding dimethyl ethanolammonium ions, and in cases excluding diethyl ethanolammonium ions). Also preferred may be N,N-dialkyl-N-[(hydroxyalkoxy)alkyl] ammonium ions, as described in connection with the first aspect of the invention.

It may be preferred for the ionic liquid of the present invention not to be any of the following compounds:
Diethanolammonium bromide
Diethanolammonium iodide
Diethanolammonium formate
Diethanolammonium acetate
Diethanolammonium propanoate
Diethanolammonium propanedioate
Diethanolammonium butanoate
Diethanolammonium butenoate
Diethanolammonium butanedioate
Diethanolammonium pentanoate
Diethanolammonium pentanedioate
Diethanolammonium pentenoate
Diethanolammonium hexanoate
Diethanolammonium hexanedioate
Diethanolammonium hexenoate
Diethanolammonium heptanoate
Diethanolammonium heptanedioate
Diethanolammonium heptenoate
Diethanolammonium octanoate
Diethanolammonium octanedioate
Diethanolammonium octenoate
Diethanolammonium nonanoate
Diethanolammonium nonanedioate
Diethanolammonium nonenoate
Diethanolammonium decanoate
Diethanolammonium decanedioate
Diethanolammonium decenoate
Diethanolammonium undecanoate
Diethanolammonium undecanedioate
Diethanolammonium undecenoate
Diethanolammonium dodecanoate
Diethanolammonium dodecanedicarboxylate
Diethanolammonium dodecenecarboxylate
Diethanolammonium cyclohexanecarboxylate
Diethanolammonium cyclohexenecarboxylate
Diethanolammonium phenoxide
Diethanolammonium benzoate
Diethanolammonium benezenedicarboxylate
Diethanolammonium benzenetricarboxylate
Diethanolammonium benzenetetracarboxylate
Diethanolammonium chlorobenzoate
Diethanolammonium fluorobenzoate
Diethanolammonium pentachlorobenzoate
Diethanolammonium pentafluorobenzoate
Diethanolammonium salicylate
Diethanolammonium glycolate
Diethanolammonium lactate
Diethanolammonium pantothenate
Diethanolammonium tartrate
Diethanolammonium hydrogen tartrate
Diethanolammonium mandelate
Diethanolammonium crotonate
Diethanolammonium malate
Diethanolanimonium pyruvate
Diethanolammonium succinate
Diethanolanimonium citrate
Diethanolammonium fumarate
Diethanolammonium phenylacetate
Diethanolammonium oxalate
Diethanolammonium bis(trifluoromethylsulphonyl)imide
Diethanolammonium carbonate
Diethanolammonium hydrogen carbonate
Diethanolammonium phosphate
Diethanolammonium hydrogen phosphate
Diethanolammonium dihydrogen phosphate
Diethanolammonium methanesulphonate
Diethanolammonium trifluoromethanesulphonate
Diethanolammonium ethylenediaminetetraacetate
Diethanolammonium hexafluorophosphate
Diethanolammonium tetrafluoroborate
Diethanolammonium trifluoroacetate
Diethanolammonium pentafluoropropanoate
Diethanolammonium heptafluorobutanoate
Diethanolammonium phosphoenolpyruvate
Diethanolammonium nicotinamide adenine dinucleotide phosphate
Diethanolammonium adenosinephosphate
Diethanolammonium adenosine diphosphate
Diethanolammonium adenosine triphosphate
Diethanolammonium oxyniacate
Diethanolammonium nitrate
Diethanolammonium nitrite
N-Butyldiethanolammonium chloride
N-Butyldiethanolammonium bromide
N-Butyldiethanolammonium iodide
N-Butyldiethanolammonium formate
N-Butyldiethanolammonium acetate
N-Butyldiethanolammonium propanoate
N-Butyldiethanolammonium propanedioate
N-Butyldiethanolammonium butanoate
N-Butyldiethanolammonium butenoate
N-Butyldiethanolammonium butanedioate
N-Butyldiethanolammonium pentanoate
N-Butyldiethanolammonium pentanedioate
N-Butyldiethanolammoniutn pentenoate
N-Butyldiethanolammonium hexanoate
N-Butyldiethanolammonium hexenoate
N-Butyldiethanolammonium heptanoate
N-Butyldiethanolammonium heptanedioate
N-Butyldiethanolammonium heptenoate
N-Butyldiethanolammonium octanoate
N-Butyldiethanolammonium octanedioate
N-Butyldiethanolammonium octenoate
N-Butyldiethanolammonium nonanoate
N-Butyldiethanolammonium nonanedioate
N-Butyldiethanolammonium nonenoate
N-Butyldiethanolammonium decanoate
N-Butyldiethanolammonium decanedioate
N-Butyldiethanolammonium decenoate
N-Butyldiethanolammonium undecanoate
N-Butyldiethanolammonium undecanedioate
N-Butyldiethanolammonium undecenoate
N-Butyldiethanolammonium dodecanoate
N-Butyldiethanolammonium dodecanedicarboxylate
N-Butyldiethanolammonium dodecenecarboxylate
N-Butyldiethanolammonium cyclohexanecarboxylate
N-Butyldiethanolammonium cyclohexenecarboxylate
N-Butyldiethanolammonium phenoxide
N-Butyldiethanolammonium benzoate
N-Butyldiethanolammonium benezenedicarboxylate
N-Butyldiethanolammonium benzenetricarboxylate
N-Butyldiethanolammonium benzenetetracarboxylate
N-Butyldiethanolammonium chlorobenzoate
N-Butyldiethanolammonium fluorobenzoate
N-Butyldiethanolammonium pentachlorobenzoate
N-Butyldiethanolammonium pentafluorobenzoate
N-Butyldiethanolammonium salicylate
N-Butyldiethanolammonium glycolate
N-Butyldiethanolammonium lactate
N-Butyldiethanolammonium pantothenate
N-Butyldiethanolammonium tartrate
N-Butyldiethanolammonium hydrogen tartrate
N-Butyldiethanolammonium mandelate
N-Butyldiethanolammonium crotonate
N-Butyldiethanolammonium malate
N-Butyldiethanolammonium pyruvate
N-Butyldiethanolammonium succinate
N-Butyldiethanolammonium citrate
N-Butyldiethanolammonium fumarate
N-Butyldiethanolammonium phenylacetate
N-Butyldiethanolammonium oxalate
N-Butyldiethanolammonium bis(trifluoromethylsulphonyl)imide
N-Butyldiethanolammonium carbonate
N-Butyldiethanolammonium hydrogen carbonate
N-Butyldiethanolammonium sulphate
N-Butyldiethanolammonium hydrogen sulphate
N-Butyldiethanolammonium phosphate
N-Butyldiethanolammonium hydrogen phosphate
N-Butyldiethanolammonium dihydrogen phosphate
N-Butyldiethanolammonium methanesulphonate
N-Butyldiethanolammonium trifluoromethanesulphonate
N-Butyldiethanolammonium ethylenediaminetetraacetate
N-Butyldiethanolammonium hexafluorophosphate
N-Butyldiethanolammonium tetrafluoroborate
N-Butyldiethanolammonium trifluoroacetate
N-Butyldiethanolammonium pentafluoropropanoate
N-Butyldiethanolammonium heptafluorobutanoate
N-Butyldiethanolammonium phosphoenolpyruvate
N-Butyldiethanolammonium nicotinamide adenine dinucleotide phosphate
N-Butyldiethanolammonium adenosinephosphate
N-Butyldiethanolammonium adenosine diphosphate
N-Butyldiethanolammonium adenosine triphosphate
N-Butyldiethanolammonium oxyniacate
N-Butyldiethanolammonium nitrate
N-Butyldiethanolammonium nitrite
N,N-Dimethylethanolammonium bromide
N,N-Dimethylethanolammonium iodide
N,N-Dimethylethanolammonium formate
N,N-Dimethylethanolammonium acetate
N,N-Dimethylethanolammonium propanoate
N,N-Dimethylethanolammonium propanedioate
N,N-Dimethylethanolammonium butanoate
N,N-Dimethylethanolammonium butenoate
N,N-Dimethylethanolammonium butanedioate
N,N-Dimethylethanolammonium pentanoate
N,N-Dimethylethanolammonium pentanedioate
N,N-Dimethylethanolammonium pentenoate
N,N-Dimethylethanolammonium hexanoate
N,N-Dimethylethanolammonium hexenoate
N,N-Dimethylethanolammonium heptanoate
N,N-Dimethylethanolammonium heptanedioate
N,N-Dimethylethanolammonium heptenoate
N,N-Dimethylethanolammonium octanoate
N,N-Dimethylethanolammonium octanedioate
N,N-Dimethylethanolammonium octenoate
N,N-Dimethylethanolammonium nonanoate
N,N-Dimethylethanolammonium nonanedioate
N,N-Dimethylethanolammonium nonenoate
N,N-Dimethylethanolammonium decanoate
N,N-Dimethylethanolammonium decanedioate
N,N-Dimethylethanolammonium decenoate
N,N-Dimethylethanolammonium undecanoate
N,N-Dimethylethanolammonium undecanedioate
N,N-Dimethylethanolammonium undecenoate
N,N-Dimethylethanolammonium dodecanoate
N,N-Dimethylethanolammonium dodecanedicarboxylate
N,N-Dimethylethanolammonium dodecenecarboxylate
N,N-Dimethylethanolammonium cyclohexanecarboxylate
N,N-Dimethylethanolammonium cyclohexenecarboxylate
N,N-Dimethylethanolammonium phenoxide
N,N-Dimethylethanolammonium benzoate
N,N-Dimethylethanolammonium benezenedicarboxylate
N,N-Dimethylethanolammonium benzenetricarboxylate
N,N-Dimethylethanolammonium benzenetetracarboxylate
N,N-Dimethylethanolammonium chlorobenzoate
N,N-Dimethylethanolammonium fluorobenzoate
N,N-Dimethylethanolammonium pentachlorobenzoate
N,N-Dimethylethanolammonium pentafluorobenzoate
N,N-Dimethylethanolammonium salicylate
N,N-Dimethylethanolammonium glycolate
N,N-Dimethylethanolammonium lactate
N,N-Dinethylethanolammonium pantothenate
N,N-Dimethylethanolammonium tartrate
N,N-Dimethylethanolammonium hydrogen tartrate
N,N-Dimethylethanolammonium mandelate
N,N-Dimethylethanolammonium crotonate
N,N-Dimethylethanolammonium malate
N,N-Dimethylethanolammonium pyruvate
N,N-Dimethylethanolammonium succinate
N,N-Dimethylethanolammonium citrate
N,N-Dimethylethanolammonium fumarate
N,N-Dimethylethanolammonium phenylacetate
N,N-Dimethylethanolammonium oxalate
N,N-Dimethylethanolammonium bis(trifluoromethylsulphonyl)imide
N,N-Dimethylethanolammonium carbonate
N,N-Dinethylethanolanmonium hydrogen carbonate
N,N-Dimethylethanolammonium sulphate
N,N-Dimethylethanolammonium hydrogen sulphate
N,N-Dimethylethanolammonium phosphate
N,N-Dimethylethanolammonium hydrogen phosphate
N,N-Dimethylethanolammonium dihydrogen phosphate
N,N-Dimethylethanolammonium methanesulphonate
N,N-Dimethylethanolammonium trifluoromethanesulphonate
N,N-Dimethylethanolammonium ethylenediaminetetraacetate
N,N-Dinethylethanolammonium hexafluorophosphate
N,N-Dimethylethanolammonium tetrafluoroborate
N,N-Dimethylethanolammonium trifluoroacetate
N,N-Dimethylethanolammonium pentafluoropropanoate
N,N-Dimethylethanolammonium heptafluorobutanoate
N,N-Dimethylethanolammonium phosphoenolpyruvate
N,N-Dinethylethanolammonium nicotinamide adenine dinucleotide phosphate
N,N-Dimethylethanolammonium adenosinephosphate
N,N-Dimethylethanolammonium adenosine diphosphate
N,N-Dimethylethanolammonium adenosine triphosphate
N,N-Dimethylethanolammonium oxyniacate
N,N-Dimethylethanolammonium nitrate
N,N-Dimethylethanolammonium nitrite
N-Methylethanolammonium bromide
N-Methylethanolammonium iodide
N-Methylethanolammonium formate
N-Methylethanolammonium acetate
N-Methylethanolammonium propanoate
N-Methylethanolammonium propanedioate
N-Methylethanolamionium butanoate
N-Methylethanolammonium butenoate
N-Methylethanolammonium butanedioate
N-Methylethanolammonium pentanoate
N-Methylethanolammonium pentanedioate
N-Methylethanolammonium pentenoate
N-Methylethanolammonium hexanoate
N-Methylethanolammonium hexenoate
N-Methylethanolammonium heptanoate
N-Methylethanolammonium heptanedioate
N-Methylethanolammonium heptenoate
N-Methylethanolammonium octanoate
N-Methylethanolammonium octanedioate
N-Methylethanolammonium octenoate
N-Methylethanolammonium nonanoate
N-Methylethanolammonium nonanedioate
N-Methylethanolammonium nonenoate
N-Methylethanolammonium decanoate
N-Methylethanolammonium decanedioate
N-Methylethanolammonium decenoate
N-Methylethanolammonium undecanoate
N-Methylethanolammonium undecanedioate
N-Methylethanolammonium undecenoate
N-Methylethanolammonium dodecanoate
N-Methylethanolammonium dodecanedicarboxylate
N-Methylethanolammonium dodecenecarboxylate
N-Methylethanolammonium cyclohexanecarboxylate
N-Methylethanolammonium cyclohexenecarboxylate
N-Methylethanolammonium phenoxide
N-Methylethanolammonium benzoate
N-Methylethanolammonium benezenedicarboxylate
N-Methylethanolammonium benzenetricarboxylate
N-Methylethanolammonium benzenetetracarboxylate
N-Methylethanolammonium chlorobenzoate
N-Methylethanolammonium fluorobenzoate
N-Methylethanolammonium pentachlorobenzoate
N-Methylethanolammonium pentafluorobenzoate
N-Methylethanolammonium salicylate
N-Methylethanolammonium glycolate
N-Methylethanolammonium lactate
N-Methylethanolammonium pantothenate
N-Methylethanolammonium tartrate
N-Methylethanolammonium hydrogen tartrate
N-Methylethanolammonium mandelate
N-Methylethanolammonium crotonate
N-Methylethanolammonium malate
N-Methylethanolammonium pyruvate
N-Methylethanolammonium succinate
N-Methylethanolammonium citrate
N-Methylethanolammonium fumarate
N-Methylethanolammonium phenylacetate
N-Methylethanolammonium oxalate
N-Methylethanolammonium bis(trifluoromethylsulphonyl)imide
N-Methylethanolammonium carbonate
N-Methylethanolammonium hydrogen carbonate
N-Methylethanolammonium sulphate
N-Methylethanolammonium hydrogen sulphate
N-Methylethanolammonium phosphate
N-Methylethanolammonium hydrogen phosphate
N-Methylethanolammonium dihydrogen phosphate
N-Methylethanolammonium methanesulphonate
N-Methylethanolammonium trifluoromethanesulphonate
N-Methylethanolammonium ethylenediaminetetraacetate
N-Methylethanolammonium hexafluorophosphate
N-Methylethanolammonium tetrafluoroborate
N-Methylethanolammonium trifluoroacetate
N-Methylethanolammonium pentafluoropropanoate
N-Methylethanolammonium heptafluorobutanoate
N-Methylethanolammonium phosphoenolpyruvate
N-Methylethanolammonium nicotinamide adenine dinucleotide phosphate
N-Methylethanolammonium adenosinephosphate
N-Methylethanolammonium adenosine diphosphate
N-Methylethanolammonium adenosine triphosphate
N-Methylethanolammonium oxyniacate
N-Methylethanolammonium nitrate
N-Methylethanolammonium nitrite
1-(3-Hydroxypropyl)putrescinium chloride
1-(3-Hydroxypropyl)putrescinium bromide
1-(3-Hydroxypropyl)putrescinium iodide
1-(3-Hydroxypropyl)putrescinium formate
1-(3-Hydroxypropyl)putrescinium acetate
1-(3-Hydroxypropyl)putrescinium propanoate
1-(3-Hydroxypropyl)putrescinium propanedioate
1-(3-Hydroxypropyl)putrescinium butanoate
1-(3-Hydroxypropyl)putrescinium butenoate
1-(3-Hydroxypropyl)putrescinium butanedioate
1-(3-Hydroxypropyl)putrescinium pentanoate
1-(3-Hydroxypropyl)putrescinium pentanedioate
1-(3-Hydroxypropyl)putrescinium pentenoate
1-(3-Hydroxypropyl)putrescinium hexanoate
1-(3-Hydroxypropyl)putrescinium hexenoate
1-(3-Hydroxypropyl)putrescinium heptanoate
1-(3-Hydroxypropyl)putrescinium heptanedioate
1-(3-Hydroxypropyl)putrescinium heptenoate
1-(3-Hydroxypropyl)putrescinium octanoate
1-(3-Hydroxypropyl)putrescinium octanedioate
1-(3-Hydroxypropyl)putrescinium octenoate
1-(3-Hydroxypropyl)putrescinium nonanoate
1-(3-Hydroxypropyl)putrescinium nonanedioate
1-(3-Hydroxypropyl)putrescinium nonenoate
1-(3-Hydroxypropyl)putrescinium decanoate
1-(3-Hydroxypropyl)putrescinium decanedioate
1-(3-Hydroxypropyl)putrescinium decenoate
1-(3-Hydroxypropyl)putrescinium undecanoate
1-(3-Hydroxypropyl)putrescinium undecanedioate
1-(3-Hydroxypropyl)putrescinium undecenoate
1-(3-Hydroxypropyl)putrescinium dodecanoate
1-(3-Hydroxypropyl)putrescinium dodecanedicarboxylate
1-(3-Hydroxypropyl)putrescinium dodecenecarboxylate
1-(3-Hydroxypropyl)putrescinium cyclohexanecarboxylate
1-(3-Hydroxypropyl)putrescinium cyclohexenecarboxylate
1-(3-Hydroxypropyl)putrescinium phenoxide
1-(3-Hydroxypropyl)putrescinium benzoate
1-(3-Hydroxypropyl)putrescinium benezenedicarboxylate
1-(3-Hydroxypropyl)putrescinium benzenetricarboxylate
1-(3-Hydroxypropyl)putrescinium benzenetetracarboxylate
1-(3-Hydroxypropyl)putrescinium chlorobenzoate
1-(3-Hydroxypropyl)putrescinium fluorobenzoate
1-(3-Hydroxypropyl)putrescinium pentachlorobenzoate
1-(3-Hydroxypropyl)putrescinium pentafluorobenzoate
1-(3-Hydroxypropyl)putrescinium salicylate
1-(3-Hydroxypropyl)putrescinium glycolate
1-(3-Hydroxypropyl)putrescinium lactate
1-(3-Hydroxypropyl)putrescinium pantothenate
1-(3-Hydroxypropyl)putrescinium tartrate
1-(3-Hydroxypropyl)putrescinium hydrogen tartrate
1-(3-Hydroxypropyl)putrescinium mandelate
1-(3-Hydroxypropyl)putrescinium crotonate
1-(3-Hydroxypropyl)putrescinium malate
1-(3-Hydroxypropyl)putrescinium pyruvate
1-(3-Hydroxypropyl)putrescinium succinate
1-(3-Hydroxypropyl)putrescinium citrate
1-(3-Hydroxypropyl)putrescinium fumarate
1-(3-Hydroxypropyl)putrescinium phenylacetate
1-(3-Hydroxypropyl)putrescinium oxalate
1-(3-Hydroxypropyl)putrescinium bis(trifluoromethylsulphonyl)imide
1-(3-Hydroxypropyl)putrescinium methanesulphonate
1-(3-Hydroxypropyl)putrescinium trifluoromethanesulphonate
1-(3-Hydroxypropyl)putrescinium hexafluorophosphate
1-(3-Hydroxypropyl)putrescinium tetrafluoroborate
1-(3-Hydroxypropyl)putrescinium trifluoroacetate
1-(3-Hydroxypropyl)putrescinium pentafluoropropanoate
1-(3-Hydroxypropyl)putrescinium heptafluorobutanoate
1-(3-Hydroxypropyl)putrescinium phosphoenolpyruvate
1-(3-Hydroxypropyl)putrescinium nicotinamide adenine dinucleotide phosphate
1-(3-Hydroxypropyl)putrescinium adenosinephosphate
1-(3-Hydroxypropyl)putrescinium adenosine diphosphate
1-(3-Hydroxypropyl)putrescinium adenosine triphosphate
1-(3-Hydroxypropyl)putrescinium carbonate
1-(3-Hydroxypropyl)putrescinium hydrogen carbonate
1-(3-Hydroxypropyl)putrescinium sulphate
1-(3-Hydroxypropyl)putrescinium hydrogen sulphate
1-(3-Hydroxypropyl)putrescinium phosphate
1-(3-Hydroxypropyl)putrescinium hydrogen phosphate
1-(3-Hydroxypropyl)putrescinium dihydrogen phosphate
1-(3-Hydroxypropyl)putrescinium nitrate
1-(3-Hydroxypropyl)putrescinium nitrite.

Again, however, generally speaking any of the anions referred to in this list may be used as the counterion in an ionic liquid according to the invention.

According to a further aspect, the present invention provides a process for the preparation of an ionic liquid according to the invention, the process comprising the steps of:
a) providing a nitrogen-containing compound of the formula (III):

NR¹R²R³ (III)

or a nitrogen-containing compound of the formula (IV):

NR⁶R⁷R⁸ (IV)
in which R¹ to R⁸ are as defined above; and
b) neutralising the compound (III) or (IV) with an acid, preferably an acid of the formula HₘX where X and m are as defined above.

The process of the present invention can provide an economical route to the manufacture of ionic liquids since the process often involves only a single step and can use starting materials that are generally readily available.

During the process of the invention, the nitrogen atom of the amine (III) or (IV) is protonated to provide a protonated ammonium ion.

Preferably, the acid includes an anion as defined herein.

Preferably the acid anion comprises a halide, halogenated inorganic anion, nitrate, sulphate, carbonate, sulphonate, carboxylate or halogenated organic anion (eg, halogenated carboxylate).

The invention also encompasses compounds of formula (III) or (IV) and their use in the preparation of one or more ionic liquids.

The invention further provides the use of a cation (I) or (II) as defined above in a solvent for an enzyme-catalysed reaction. Further provided is the use of an ionic liquid according to the present invention as a solvent for an enzyme-catalysed reaction.

The use of ionic liquids in certain biological and/or chemical reactions can have several advantages over traditional aqueous solutions. Ionic liquids have an ability to dissolve a wide range of inorganic, organic, polymeric and biological materials, often to a very high concentration. They have a wide liquid range, allowing both high and low temperature processes to be carried out in the same solvent. They do not elicit solvolysis phenomena and most stabilise short-lived reactive intermediates. There are no pH effects in the solvents and there is practically zero vapour pressure over much of the liquid range. Ionic liquids also exhibit excellent electrical and thermal conductivity whilst being non-flammable, recyclable and generally of low toxicity.

The invention further provides the use of an ionic liquid according to the present invention in or as a solvent for organic synthesis, a matrix in matrix-assisted laser desorption/ionisation (MALDI) mass spectrometry, a solvent for a solvent extraction process (eg, to remove desired components from an immiscible liquid or solid), a vehicle in chromatography (eg, gas chromatography), a lubricant, a hydraulic fluid or a biocide. Also provided is the use of an ionic liquid according to the invention (for instance as a solvent) in catalysis, liquefaction, nuclear fuel reprocessing, fuel cells, electrochemical applications, optical (including optoelectronic) systems, pervaporation, drug delivery, adhesives or sensors.

Preferably an ionic liquid according to the invention is used as a reaction medium—preferably a solvent—for a chemical or biochemical reaction, in particular a catalysed reaction, such as an enzyme-catalysed reaction. It may be particularly suited as a solvent for materials which would otherwise require an aqueous, or at least polar and/or hydrogen bonding, solvent environment.

The invention thus further provides a method for carrying out an enzyme-catalysed reaction comprising:
a) providing a liquid reaction medium which comprises an ionic liquid according to the present invention;
b) providing in the liquid reaction medium an enzyme and a substrate for the enzyme; and
c) allowing reaction of the substrate to occur.

Further provided is a method for the synthesis of one or more organic compounds, the method comprising carrying out an organic synthesis reaction in an ionic liquid according to the present invention.

Throughout the description and claims of this specification, the words “comprise” and “contain” and variations of the words, for example “comprising” and “comprises”, mean “including but not limited to”, and are not intended to (and do not) exclude other moieties, additives, components, integers or steps.

Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics, compounds, chemical moieties or groups described in conjunction with a particular aspect, embodiment or example of the invention are to be understood to be applicable to any other aspect, embodiment or example described herein unless incompatible therewith.

Preferred features of each aspect of the invention may be as described in connection with any of the other aspects.

Other features of the present invention will become apparent from the following examples. Generally speaking the invention extends to any novel one, or any novel combination, of the features disclosed in this specification (including any accompanying claims and drawings). Moreover unless stated otherwise, any feature disclosed herein may be replaced by an alternative feature serving the same or a similar purpose.

The present invention will now be further described with reference to the following non-limiting examples.

### EXAMPLE 1

### Preparation of N-(methoxypropyl)ammonium Glycolate

Glycolic acid (76.05 g) and 3-methoxypropylamine (89.14 g, 1 equiv.) were independently dissolved in 200 mL volumes of absolute ethanol. The acid solution was added dropwise to the amine over a period of 1 hour, with magnetic stirring and external cooling being continued throughout. At the conclusion of the reaction the solvent was removed in vacuo and the product was dried by lyophilization to yield a yellow liquid, N-(methoxypropyl)ammonium glycolate.

### EXAMPLE 2

### Preparation of Further Ionic Liquids

Using an analogous method to that of Example 1, further ionic liquids in accordance with the invention were prepared and their viscosities, densities and/or refractive indices measured. Where the starting materials were both liquids, no solvent was used for the synthesis.

Viscosities were measured using an AND™ SV10 vibrational viscometer. Refractive indices were obtained using a Mettler Toledo Refracto™ 30 portable refractometer. Densities were measured simply by determining the mass of a measured volume of the liquid.

All products were dried prior to characterisation, to a water content of between about 0.2 and 1% w/w.

All products were liquid at room temperature, apart from 2-methoxyethylammonium acetate and N-butylethanolammonium propionate which were solid at room temperature but meltable at 51° C. and 25° C. respectively.

The starting materials used are shown in Table 1 below, and the properties in Table 2.

TABLE 1

| Product | Amine | Acid |
|---|---|---|
| N,N-dimethyl-2-methoxyethylammonium acetate | N,N-dimethyl-2- | Acetic |
| | methoxyethylamine | |
| N,N-dimethyl-2-methoxyethylammonium | N,N-dimethyl-2- | Propanoic |
| propionate | methoxyethylamine | |
| N,N-dimethyl-2-methoxyethylammonium | N,N-dimethyl-2- | Octanoic |
| octanoate | methoxyethylamine | |
| N,N-dimethyl-2-methoxyethylammonium | N,N-dimethyl-2- | Glycolic |
| glycolate | methoxyethylamine | |
| N,N-dimethyl-2-methoxyethylammonium | N,N-dimethyl-2- | Methane sulphonic |
| methanesulphonate | methoxyethylamine | |
| N,N-dimethyl-2-methoxyethylammonium | N,N-dimethyl-2- | Ethane sulphonic |
| ethanesulphonate | methoxyethylamine | |
| N,N-dimethyl-2-methoxyethylammonium | N,N-dimethyl-2- | Sulphamic |
| sulphamate | methoxyethylamine | |
| N-methyl-bis(2-methoxyethyl)ammonium acetate | N-methyl-bis(2- | Acetic |
| | methoxyethyl)amine | |
| N-methyl-bis(2-methoxyethyl)ammonium | N-methyl-bis(2- | Propanoic |
| propionate | methoxyethyl)amine | |
| N-methyl-bis(2-methoxyethyl)ammonium | N-methyl-bis(2- | Octanoic |
| octanoate | methoxyethyl)amine | |
| N-methyl-bis(2-methoxyethyl)ammonium | N-methyl-bis(2- | Glycolic |
| glycolate | methoxyethyl)amine | |
| N-methyl-bis(2-methoxyethyl)ammonium | N-methyl-bis(2- | Methane sulphonic |
| methanesulphonate | methoxyethyl)amine | |
| 3-methoxypropylammonium acetate | 3-methoxypropylamine | Acetic |
| 3-methoxypropylammonium glycolate | 3-methoxypropylamine | Glycolic |
| N,N-diethylethanolammonium formate | N,N-diethylethanolamine | Formic |
| N,N-diethylethanolammonium acetate | ″ | Acetic |
| N,N-diethylethanolammonium propionate | ″ | Propanoic |
| N,N-diethylethanolammonium octanoate | ″ | Octanoic |
| N,N-diethylethanolammonium glycolate | ″ | Glycolic |
| N,N-diethylethanolammonium methanesulphonate | ″ | Methane sulphonic |
| N,N-diethylethanolammonium ethanesulphonate | ″ | Ethane sulphonic |
| N,N-diethylethanolammonium sulphamate | ″ | Sulphamic |
| N,N-diethylethanolammonium | ″ | Bis(trifluoro |
| bis(trifluoromethanesulphonyl)imide | | methanesulphonyl) |
| | | imidic acid |
| N-propylethanolammonium acetate | N-propylethanolamine | Acetic |
| N-propylethanolammonium propionate | ″ | Propanoic |
| N-butylethanolammonium formate | N-butylethanolamine | Formic |
| N-butylethanolammonium acetate | ″ | Acetic |
| N-butylethanolammonium propionate | ″ | Propanoic |
| N-butylethanolammonium octanoate | ″ | Octanoic |
| N-butylethanolammonium glycolate | ″ | Glycolic |
| N,N-dimethylpropanolammonium formate | N,N-dimethylpropanolamine | Formic |
| N,N-dimethylpropanolammonium acetate | ″ | Acetic |
| N,N-dimethylpropanolammonium propionate | ″ | Propanoic |
| N,N-dimethylpropanolammonium glycolate | ″ | Glycolic |
| N,N-diethylpropane-2,3-diolammonium acetate | N,N-diethylpropane-2,3-diolamine | Acetic |
| N,N-dimethyl-N- [(2- | N,N-dimethyl-N-[(2- | Acetic |
| hydroxyethoxy)ethyl]ammonium acetate | hydroxyethoxy)ethyl]amine | |

Most of the starting materials were readily available as off-the-shelf products. N,N-dimethyl-2-methoxyethylamine and N-methyl-bis(2-methoxyethyl)amine were sourced from CSS Chemicals, Belfast, Northern Ireland.

TABLE 2

| | Viscosity at | | |
|---|---|---|---|
| | 25° C. | Refractive | Density |
| | (centipoise) | index | (g/cm³) |
| | (error ± | (error ± | (error ± |
| Product | ~5%) | ~2%) | ~2%) |
| N,N-dimethyl-2-methoxyethylammonium acetate | | 1.426 | |
| N,N-dimethyl-2-methoxyethylammonium propionate | | 1.422 | |
| N,N-dimethyl-2-methoxyethylammonium octanoate | 13.5 | 1.429 | 0.93 |
| N,N-dimethyl-2-methoxyethylammonium glycolate | | 1.459 | |
| N,N-dimethyl-2-methoxyethylammonium | | 1.449 | |
| methanesulphonate | | | |
| N,N-dimethyl-2-methoxyethylammonium | | 1.449 | |
| ethanesulphonate | | | |
| N,N-dimethyl-2-methoxyethylammonium | | 1.472 | |
| sulphamate | | | |
| N-methyl-bis(2-methoxyethyl)ammonium acetate | | 1.423 | |
| N-methyl-bis(2-methoxyethyl)ammonium propionate | | 1.423 | |
| N-methyl-bis(2-methoxyethyl)ammonium octanoate | | 1.431 | |
| N-methyl-bis(2-methoxyethyl)ammonium glycolate | | 1.452 | |
| 3-methoxypropylammonium acetate | 640 | 1.447 | 1.04 |
| 3-methoxypropylammonium glycolate | 2670 | 1.470 | 1.17 |
| N,N-diethylethanolammonium formate | 395 | 1.462 | 1.04 |
| N,N-diethylethanolammonium acetate | 55 | 1.453 | 1.00 |
| N,N-diethylethanolammonium propionate | 42 | 1.450 | 0.98 |
| N,N-diethylethanolammonium octanoate | 46 | 1.451 | 0.96 |
| N,N-diethylethanolammonium glycolate | 740 | 1.466 | 1.11 |
| N-propylethanolammonium acetate | 1310 | 1.459 | 1.04 |
| N-propylethanolammonium propionate | 483 | 1.456 | 1.02 |
| N-butylethanolammonium formate | 376 | 1.462 | 1.04 |
| N-butylethanolammonium acetate | 365 | 1.455 | 1.04 |
| N-butylethanolammonium propionate | | 1.457 | 1.00 |
| N-butylethanolammonium octanoate | 388 | 1.456 | |
| N-butylethanolammonium glycolate | 800 | 1.464 | 1.08 |
| N,N-dimethylpropanolammonium formate | 132 | 1.460 | 1.07 |
| N,N-dimethylpropanolammonium acetate | 77 | 1.451 | 1.03 |
| N,N-dimethylpropanolammonium propionate | 52 | 1.441 | 0.99 |
| N,N-dimethylpropanolammonium glycolate | 3320 | 1.472 | 1.14 |
| N,N-diethylpropane-2,3-diolammonium acetate | 3100 | | |
| N,N-dimethyl-N-[(2- | 48 | 1.45 | 1.05 |
| hydroxyethoxy)ethyl]ammonium acetate | | | |

### Example 3

### Use of the Ionic Liquids in Biocatalysis

Ionic liquids according to the present invention, such as those described in Examples 1 and 2, may be used as reaction media for enzyme-catalysed reactions. For example, an ionic liquid such as an alkyl alkoxyalkyl ammonium salt, a dialkyl alkoxyalkyl ammonium salt or an alkyl di(alkoxyalkyl) ammonium salt may be used as a solvent for a reaction catalysed by a hydrolase or an esterase. Generally speaking, it will be possible to use such an ionic liquid as the sole solvent, without the need for an aqueous cosolvent in order to preserve enzyme activity.

Similarly, ionic liquids such as alkyl alkanolammonium salts and dialkyl alkanolammonium salts may be used as solvents in biocatalysis.

### EXAMPLE 4

### Further Uses of the Ionic Liquids

Ionic liquids according to the invention may be used as solvents in a wide range of situations, including as reaction media for both chemical and biochemical (including enzyme-catalysed) reactions, or as extracting solvents for target solutes. By varying the nature of the substituents on the central nitrogen atom, the solvating properties, viscosity, melting point and other relevant properties of the ionic liquid can be varied according to requirements, thus offering the opportunity to “tailor” the ionic liquid as a solvent for a specific solute or solutes.

For example, if the ionic liquid is to be used as a solvent in an environment containing an activated acid or a strong base, then it may be preferred not to include hydroxyl groups on the cation—in such a situation, cations substituted with only alkyl and alkoxyalkyl groups may then be appropriate. The same may apply when the ionic liquid is to be used as a medium for a hydrolase- or esterase-catalysed reaction.

If the ionic liquid is to be used as a solvent for a metal-containing species, then it may be preferred for the cation to be substituted with two alkoxyalkyl groups, as it can then act as a chelating agent and help to solubilise the metal-containing species.

If the ionic liquid is to be used to dissolve a cellulosic material, then a cation substituted with a group of formula —(CH₂)ₙ—O—(CH₂)ₘ—OH (where n and m are independently selected integers, suitably from 2 to 4) may be preferred, for instance an N,N-dialkyl-N-[(2-hydroxyethoxy)ethyl] ammonium ion, in particular an N,N-dimethyl-N-[(2-hydroxyethoxy)ethyl] ammonium ion.

1. An ionic liquid comprising a cation of the formula (I):
N⁺HR¹R²R³ (I)
wherein R¹ is a group —R⁴—O—R⁵;
R² and R³ are each independently either hydrogen or hydrocarbyl, or R² and R³ may be joined together with the N to form a heterocyclic group;
R⁴ is a divalent hydrocarbyl radical; and
R⁵ is hydrocarbyl.

2. An ionic liquid according to claim 1, wherein the cation is a secondary or tertiary ammonium ion.

3. An ionic liquid according to claim 1, wherein R⁴ is —(CH₂)ₙ—, and where n is an integer from 2 to 4.

4. An ionic liquid according to claim 3, wherein n is 2.

5. An ionic liquid according to claim 1, wherein R⁵ is C₁ to C₄ alkyl.

6. An ionic liquid according to claim 5, wherein R⁵ is methyl.

7. An ionic liquid according to claim 1 4, wherein R⁵ is (CH₂)ₙOH, and n is an integer from 1 to 4.

8. An ionic liquid according to claim 1, wherein R² is C₁ to C₄ alkyl.

9. An ionic liquid according to claim 8, wherein R² is methyl.

10. An ionic liquid according to claim 1, wherein R² is —R⁴—O—R⁵, R⁴ is —(CH₂)ₙ— and n is an integer from 2 to 4, R⁵ is either C₁ to C₄ alkyl or (CH₂)ₙOH and n is an integer from 1 to 4, and wherein R² is either the same as or different to R¹.

11. An ionic liquid according to claim 1, wherein R³ is hydrogen.

12. An ionic liquid according to claim 1, wherein R² and R³ are each independently selected from C₁ to C₃ alkyl groups.

13. An ionic liquid according to claim 12, wherein R² and R³ are both methyl.

14. An ionic liquid according to claim 1, wherein either or both of R² and R³ is an alkanolyl group.

15. An ionic liquid according to claim 14, wherein R² is an alkanolyl group and R³ is an alkyl group.

16. An ionic liquid according to claim 14, wherein the alkanolyl group is a C₂ to C₅ alkanolyl group.

17. An ionic liquid according to claim 16, wherein the alkanolyl group is ethanolyl.

18. An ionic liquid according to claim 1, wherein the cation (I) is selected from the group consistinq of an alkoxypropyl ammonium cation, a methoxyalkyl ammonium cation, a di(alkoxyalkyl) ammonium cation other than a di(methoxyethyl) ammonium cation, an alkyl alkoxyalkyl ammonium cation, a dialkyl alkoxyalkyl ammonium cation, an alkyl di(alkoxyalkyl) ammonium cation and a N,N-dialkyl-N-[(hydroxyalkoxy)alkyl] ammonium cation.

19. An ionic liquid according to claim 18, wherein the cation (I) is selected from the group consisting of an alkyl alkoxyalkyl ammonium cation, a dialkyl alkoxyalkyl ammonium cation, an alkyl di(alkoxyalkyl) ammonium cation and a N,N-dialkyl-N-[(hydroxyalkoxy)alkyl] ammonium cation.

20. An ionic liquid according to claim 19, wherein the cation (I) is selected from the group consisting of a dialkyl alkoxyalkyl ammonium cation, an alkyl di(alkoxyalkyl) ammonium cation and a N,N-dialkyl-N-[(hydroxyalkoxy)alkyl] ammonium cation.

21. An ionic liquid according to claim 20, wherein the cation (I) is a dimethyl 2-methoxyethyl ammonium cation or a methyl-bis(2-methoxyethyl) ammonium cation.

22. An ionic liquid according to claim 1, wherein the cation (I) is selected from the group consisting of methyl alkoxyethyl ammonium cations, methyl alkoxypropyl ammonium cations, dimethyl alkoxyethyl ammonium cations, dimethyl alkoxypropyl ammonium cations, ethyl alkoxyethyl ammonium cations, ethyl alkoxypropyl ammonium cations, diethyl alkoxyethyl ammonium cations, diethyl alkoxypropyl ammonium cations, methyl ethyl alkoxyethyl ammonium cations, methyl ethyl alkoxypropyl ammonium cations, propyl alkoxyethyl ammonium cations, propyl alkoxypropyl ammonium cations, dipropyl alkoxyethyl ammonium cations, dipropyl alkoxypropyl ammonium cations, methyl propyl alkoxyethyl ammonium cations, methyl propyl alkoxypropyl ammonium cations, ethyl propyl alkoxyethyl ammonium cations and ethyl propyl alkoxypropyl ammonium cations.

23. An ionic liquid according to claim 22, wherein the cation (I) is selected from the group consisting of alkoxypropyl ammonium, methyl alkoxyethyl ammonium, methyl alkoxypropyl ammonium, dimethyl alkoxyethyl ammonium and ethyl methyl alkoxyethyl ammonium ions.

24. An ionic liquid according to claim 22 or claim 23, wherein the alkoxy group is either methoxy or ethoxy.

25. An ionic liquid comprising a cation of the formula (II):
N⁺HR⁶R⁷R⁸ (II)
wherein R⁶ is an alkanolyl group;
R⁷ is a hydrocarbyl group; and
R⁸ is either hydrogen or hydrocarbyl, or R⁷ and R⁸ may be joined together with the N to form a heterocyclic group.

26. An ionic liquid according to claim 25, wherein R⁶ is selected from the group consisting of methanolyl, ethanolyl and propanolyl.

27. An ionic liquid according to claim 26, wherein R⁶ is ethanolyl or propanolyl.

28. An ionic liquid according to claim 25, wherein R⁶ is a —(CH₂)ₙ—O—(CH₂)ₘ—OH where n and m are independently selected integers from 1 to 4.

29. An ionic liquid according to claim 25, wherein R⁶ contains a terminal —OH group.

30. An ionic liquid according to claim 25, wherein R⁶ and R⁷ are each independently selected from alkanolyl groups.

31. An ionic liquid according to claim 25, wherein R⁷ is C₁ to C4 alkyl.

32. An ionic liquid according to claim 25, wherein R⁸ is C₁ to C₄ alkyl.

33. An ionic liquid according to claim 25, wherein R⁷ and R⁸ are each independently selected from methyl and ethyl.

34. An ionic liquid according to claim 25, wherein R⁸ is hydrogen.

35. An ionic liquid according to claim 25, wherein the cation (II) is selected from the group consisting of alkyl alkanolammonium ions, dialkyl alkanolammonium ions and N,N-dialkyl-N-[(hydroxyalkoxy)alkyl] ammonium ions.

36. An ionic liquid according to claim 35, wherein the cation (II) is selected from the group consisting of propyl ethanolammomium, butyl ethanolammomium, dimethyl propanolammonium and diethyl propanolammonium ions.

37. An ionic liquid according to claim 1, which is capable of existing in liquid form at and below 40° C.

38. An ionic liquid according to claim 37, which is capable of existing in liquid form at room temperature.

39. An ionic liquid according to claim 1, which contains 5% or less of water, by mass.

40. An ionic liquid according to claim 1, which has a viscosity of less than 500 centipoise at 25° C.

41. An ionic liquid according to claim 1, which comprises a counterion X^{m-} where m is an integer from 1 to 3.

42. (canceled)

43. A process for the preparation of an ionic liquid the process comprising the steps of:
a) providing a nitrogen-containing compound of the formula (III):
NR¹R²R³ (III)
or a nitrogen-containing compound of the formula (IV):
NR⁶R⁷R⁸ (IV)
wherein R¹ is —R⁴—O—R⁵;
R² and R³ are each independently either hydrogen or hydrocarbyl, or R² and R³ may be joined together with the N to form a heterocyclic group;
R⁴ is a divalent hydrocarbyl radical;
R⁵ is hydrocarbyl;
R⁶ is an alkanolyl group;
R⁷ is a hydrocarbyl group;
R⁸ is either hydrogen or hydrocarbyl, or R⁷ and R⁸ may be joined together with the N to form a heterocyclic group; and
b) neutralising the compound (III) or (IV) with an acid.

44. A process according to claim 43, wherein the acid used in step (b) has the formula HₘX where m is an integer from 1 to 3 and the anion X is selected from the group consisting of a halide, halogenated inorganic anion, nitrate, sulphate, carbonate, sulphonate, carboxylate and halogenated organic anion.

45 (canceled)

46 (canceled)

47 (canceled)
